(19) United States
(12) Patent Application Publication
(10) Pub. No.: US 2024/0301324 A1
(43) Pub. Date: Sep. 12, 2024

(54) COMPOSITION

(71) Applicant: Croda International Plc, Goole, Yorkshire (GB)

(72) Inventors: Katy HELM, Goole, Yorkshire (GB); Heather CLARKSON, Goole, Yorkshire (GB); Timothy A. MILLER, Goole, Yorkshire (GB)

(73) Assignee: Croda International Plc, Goole, Yorkshire (GB)

(21) Appl. No.: 18/578,597

(22) PCT Filed: Aug. 5, 2022
(86) PCT No.: PCT/EP2022/072112

§ 371 Date: Jan. 11, 2024

(30) Foreign Application Priority Data
Aug. 9, 2021 (GB) 2111457.4

(51) Int. Cl. C11D 1/94; A61K 8/37; A61K 8/44; A61K 8/46; A61Q 5/02; A61Q 19/10; C11D 1/14; C11D 1/83

(52) U.S. Cl. CPC C11D 1/94; A61K 8/37; A61K 8/44; A61K 8/463; A61Q 5/02; A61Q 19/10; C11D 1/146; C11D 1/83; A61K 2800/30; A61K 2800/596

(57) ABSTRACT

The present invention relates to the use of a combination of a sophorolipid surfactant and an zwitterionic surfactant selected from the group consisting of betaines and hydroxysultaines in a composition to improve the mildness to skin of the composition by reducing the amount of zein protein solubilised by the composition, measured as described herein, wherein the composition comprises an anionic surfactant. The invention also relates to a method of reducing the amount of zein protein solubilised by a composition comprising an anionic surfactant, the method comprising the steps of: a) including a sophorolipid surfactant in the composition; and b) including a zwitterionic surfactant selected from the group consisting of betaines and hydroxysultaines in the composition; wherein the amount of zein protein solubilised by the composition is reduced by at least 20 wt %, measured as described herein. The invention further relates to a composition comprising sophorolipid surfactant, anionic surfactant and zwitterionic surfactant.

### FIELD OF THE INVENTION

The present invention relates to a composition comprising a sophorolipid surfactant, an anionic surfactant and a zwitterionic surfactant and also to home care and personal care formulations comprising the composition. The invention also relates to use of the combination of a sophorolipid surfactant and a zwitterionic surfactant to improve the mildness to skin of a composition comprising an anionic surfactant by reducing the amount of zein protein solubilised by the composition.

### BACKGROUND

Surfactants are amphiphilic molecules which are able to reduce interfacial and surface tension in formulations. They are commonly used within the home care and personal care industry due to their ability to remove grease and dirt from the site of application which is useful for detergents including laundry detergents, hair shampoos and soaps. Interest in the use of sophorolipids as biosurfactants has grown recently due to their improved environmental profile when compared with petrochemical derived surfactants. Many consumers are becoming more aware of what ingredients are used in their home care and personal care products and are selecting products based on their environmental impact. This makes sophorolipids a good alternative to synthetic surfactants since they are synthesised by culturing yeasts and are able to biodegrade, thus they are ‘green’ ingredients.

U.S. Pat. No. 8,563,490 discloses the use of a mixture of 1 to 20 wt % sophorolipids and 1 to 20 wt % anionic surfactant which must be selected from the group consisting of glycinate, sulphosuccinate, and mixtures thereof in a detergent composition to increase the mildness to the skin of the composition. More specifically the anionic surfactant is selected from the group consisting of cocoyl glycinate sodium salt and disodium laureth-3-sulphosuccinate. The sophorolipid used in the invention is Sopholiance S—ex Soliance. It is said to be a Candida bombicola/Glucose/Methyl Rapeseedate Ferment (and) water (and) Potassium Sorbate. Olivamidopropyl betaine may be used in the invention for foam boosting.

U.S. Pat. No. 9,271,908 discloses aqueous hair and skin cleaning compositions comprising at least one biosurfactant and at least one fatty acid. Table 1 of the document demonstrates that the invention is not concerned with the combination of biosurfactant and betaine surfactant since these are tested separately in the handwashing formulations.

There is a continual need for the development of improved home care and personal care formulations and ingredients.

### SUMMARY OF THE INVENTION

It is an object of the present invention to address the above and/or other disadvantages associated with the prior art.

According to a first aspect, the present invention provides the use of a combination of a sophorolipid surfactant and an zwitterionic surfactant selected from the group consisting of betaines and hydroxysultaines in a composition to improve the mildness to skin of the composition by reducing the amount of zein protein solubilised by the composition, measured as described herein, wherein the composition comprises an anionic surfactant.

The use of the first aspect may comprise any of the features of the composition of the second aspect of the invention.

According to a second aspect, the present invention provides a composition comprising:
a) sophorolipid surfactant;
b) anionic surfactant; and
c) 3 to 7 wt % zwitterionic surfactant selected from the group consisting of betaines and hydroxysultaines, wherein the wt % is on the basis of the total weight of the composition;
wherein the weight ratio of the sophorolipid surfactant to the anionic surfactant in the composition is from 1:20 to 1:2; and
wherein the composition comprises no free fatty acid.

According to a third aspect, the present invention provides a method of reducing the amount of zein protein solubilised by a composition comprising an anionic surfactant, the method comprising the steps of:
a) including a sophorolipid surfactant in the composition; and
b) including a zwitterionic surfactant selected from the group consisting of betaines and hydroxysultaines in the composition;
wherein the amount of zein protein solubilised by the composition is reduced by at least 20%, measured as described herein.

The method of the third aspect may comprise any of the features of the first or second aspects of the invention.

The present invention is based in part on the recognition by the inventors that a surprising improvement in the mildness to skin of a composition comprising an anionic surfactant can be provided by the use of a synergistic combination of a sophorolipid surfactant and a zwitterionic surfactant selected from the group consisting of betaines and hydroxysultaines in the composition. Without being bound by theory, it is believed that the combination of the sophorolipid surfactant and the zwitterionic surfactant reduces the charge effect of the anionic surfactant in the composition, and hence synergistically improves the mildness of the composition. The mildness to skin of a composition may be measured by the amount of zein protein solubilised by the composition—with a milder composition solubilising less zein protein.

As demonstrated herein, the combination of a sophorolipid and cocamidopropyl betaine or cocamidopropyl hydroxysultaine can synergistically reduce the amount of zein protein solubilised by a composition comprising an anionic surfactant such as sodium lauryl ether sulphate or sodium lauryl sarcosinate.

All of the features described herein may be present in any of the above aspects of the invention, in any combination.

### DETAILED DESCRIPTION OF THE INVENTION

It will be understood that any upper or lower quantity or range limit used herein may be independently combined.

It will be understood that, when describing the number of carbon atoms in a substituent group (e.g. ‘C1 to C6’), the number refers to the total number of carbon atoms present in the substituent group, including any present in any branched groups. Additionally, when describing the number of carbon atoms in, for example fatty acids, this refers to the total number of carbon atoms including the one at the carboxylic acid, and any present in any branch groups.

The term ‘free fatty acid’ means a fatty acid, for example oleic acid, which is present as itself and not reacted with another moiety to form a compound. The term ‘no free fatty acid’ means free fatty acid is not present in substantial amounts, such as preferably the composition of the invention comprises less than 5 wt % fatty acid, preferably comprises less than 4 wt % fatty acid, preferably comprises less than 3 wt % fatty acid, preferably comprises less than 2 wt % fatty acid, preferably comprises less than 1 wt % fatty acid, all on the basis of the total weight of the composition. Alternatively, the composition of the invention may comprise less than 25 grams of free fatty acid per litre (g/L), preferably less than 20 g/L, preferably less than 15 g/L, preferably less than 10 g/L on the basis of the total weight of free fatty acid and the total volume of the composition.

The term ‘personal care formulation’ means a product intended to be applied to the human body or any part thereof for cleansing, beautifying or improving appearance. Personal care formulations include but are not limited to hand soaps; bar soaps; liquid soaps; facial and body washes; facial and body cleansers; shampoos; conditioners; toothpaste; shaving creams or gels; and foot care products. A personal care formulation does not include any product for which a prescription is required.

The term ‘home care formulation’ means a product used by household and institutional consumers for cleaning, caring or conditioning of the home or any of its contents including, but not limited to, detergents including laundry detergents and dishwashing detergents; cleaning compounds including hard surface cleaners; polishes and floor finishes.

The term ‘hair care formulation’ means a product used for the care of human hair. The term ‘hair care ingredient’ means an ingredient suitable for and intended to be applied to human hair.

The term ‘skin care formulation’ means a product used for the care of human skin. The term ‘skin care ingredient’ means an ingredient suitable for and intended to be applied to human skin.

Mildness to skin is determined herein by measuring zein protein solubilisation to provide an indication of the ability of a surfactant to solubilise the proteins within the top layer of the Stratum Corneum. A high percentage of zein protein solubilised correlates to a harsh surfactant on the skin, whereas a low percentage of zein protein solubilised correlates to a mild surfactant. Thus a reduction in zein protein solubilisation corresponds to an improvement in mildness.

### Composition According to the Invention

A composition according to the invention comprises:
a) sophorolipid surfactant;
b) anionic surfactant; and
c) 3 to 7 wt % zwitterionic surfactant selected from the group consisting of betaines and hydroxysultaines, wherein the wt % is on the basis of the total weight of the composition;
wherein the weight ratio of the sophorolipid surfactant to the anionic surfactant in the composition is from 1:20 to 1:2; and
wherein the composition comprises no free fatty acid.

### Sophorolipid Surfactant

The sophorolipid surfactant may be obtainable, preferably is obtained by a fermentation process. The sophorolipid may be obtainable from a natural or modified micro-organism, preferably selected from the group of Starmerella (or Candida) bombicola, Candida albicans, Wickerhamiella domericqiae, Candida apicola, Candida floricola, Candida kuoi, Candida stellata, Candida riodocensis, Candida batistae, Candida tropicalis, Cyberlindera samutprakarnensis. Preferably the sophorolipid is obtainable from Starmerella bombicola.

Sophorolipids are part of the bio-surfactant family. The term ‘bio’ refers to their method of production by biotechnology e.g. by fermentation. The fermentation process typically utilises sugars and alkanes or lipids as substrates. Appropriate fermentation methods are reviewed in A P Tulloch, J F T Spencer and P A J Gorin, Can. J Chem (1962) 40 1326 and U Gobbert, S Lang and F Wagner, Biotechnology Letters (1984) 6 (4), 225.

Commercially available sophorolipids may be used in the invention. Examples of such sophorolipids are produced by the company WHEATOLEO under the commercial name “SOPHOCLEAN” and by the company GIVAUDAN under the commercial name “SOPHOLIANCE S”.

In a sophorolipid obtainable from Starmerella bombicola, the hydrophilic moiety of the biosurfactant molecule is a disaccharide (i.e., sophorose), and the hydrophobic portion is an omega- or (omega-1)-hydroxy fatty acid attached to the sophorose via a glycosidic bond. The fatty acid chain, most commonly containing 16- and 18-carbon atoms, may be unsaturated and lactonized to the disaccharide. There are generally considered to be two types of sophorolipids, the acid form (open-chain) and the lactone form (closed-chain) sophorolipids. To produce a lactone form sophorolipid, the hydroxyl fatty acid moiety of the acid form sophorolipids forms a macrocyclic lactone ring with the 4″-hydroxyl group of the sophorose by intramolecular esterification.

Preferably the sophorolipid of the invention is a mixture of acid form (open-chain) sophorolipids and lactone form (closed-chain) sophorolipids. The sophorolipid preferably comprises at least 40 wt %, more preferably at least 50 wt %, particularly preferably at least 55 wt %, possibly at least 65 wt %, potentially at least 75 wt % of acid form sophorolipid on the basis of the total weight of sophorolipid in the composition. The sophorolipid preferably comprises at most 60 wt %, more preferably at most 50 wt %, particularly preferably at most 45 wt %, possibly at most 35 wt %, potentially at most 25 wt % lactone form sophorolipid on the basis of the total weight of sophorolipid in the composition. Preferably the sophorolipid surfactant comprises at least 55 wt % acid form sophorolipid and at most 45 wt % lactone form sophorolipid on the basis of the total weight of sophorolipid in the composition.

The composition may comprise at least 1 wt % sophorolipid surfactant, preferably at least 2 wt %, more preferably at least 4 wt %, particularly preferably at least 5 wt % wherein the wt % is on the basis of the total weight of the composition. The composition may comprise at most 15 wt % sophorolipid surfactant, preferably at most 12 wt %, more preferably at most 10 wt %, particularly preferably at most 8 wt % wherein the wt % is on the basis of the total weight of the composition.

### Anionic Surfactant

The anionic surfactant may be selected from the group consisting of alkyl sulphates, alkyl ether sulphates, alkyl amido ether sulphates, alkylsulfonates, alkylphosphates, alkylamidesulfonates, alkylarylsulfonates, alkylsulfosuccinates, alkylethersulfosuccinates, alkylamidesulfosuccinates; alkylsulfosuccinamates; alkylsulfoacetates; alkyl ether phosphates; sarcosinates; taurates and isethionates.

Preferably the anionic surfactant is selected from the group consisting of alkyl sulphates, alkyl ether sulphates and sarcosinates.

Preferably the anionic surfactant comprises at least one of sodium lauryl sulphate, sodium lauryl ether sulphate and sodium lauryl sarcosinate.

Preferably the anionic surfactant comprises a sarcosinate selected from sodium lauroyl sarcosinate, sodium myristoyl sarcosinate, potassium myristoyl sarcosinate, sodium cocoyl sarcosinate, sodium oleyl sarcosinate, ammonium oleyl sarcosinate and triethanolamine lauroyl sarcosinate, and mixtures thereof. Preferably, the sarcosinate is sodium lauroyl sarcosinate.

Preferably the composition does not comprise a glycinate surfactant, a sulphosuccinate surfactant, a fatty acyl isethionate or mixtures thereof.

Preferably the anionic surfactant does not comprise a sulphate containing compound.

The composition may comprise at least 1 wt % anionic surfactant, preferably at least 2 wt %, more preferably at least 4 wt %, particularly preferably at least 5 wt % wherein the wt % is on the basis of the total weight of the composition. The composition may comprise at most 25 wt % anionic surfactant, preferably at most 20 wt %, more preferably at most 18 wt %, particularly preferably at most 15 wt % wherein the wt % is on the basis of the total weight of the composition.

### Zwitterionic Surfactant

The zwitterionic surfactant is selected from the group consisting of betaines and hydroxysultaines. A zwitterionic surfactant is a surfactant that carries both a positive and a negative charge. These charges can either be permanent or dependent on the pH value.

The composition comprises 3 to 7 wt % zwitterionic surfactant selected from the group consisting of betaines and hydroxysultaines, wherein the wt % is on the basis of the total weight of the composition.

Betaines which may be included in the composition may be selected from dodecylbetaine, tetradecylbetaine, hexadecylbetaine, laurylamidopropyl betaine and cocamidopropyl betaine

Examples of suitable betaines are carboxyalkylation products of amidoamines. Particularly suitable are amidopropyl betaines. Preferred amidopropyl betaines are cocamidopropyl betaine (coco is the fatty acid radical of coconut oil, chain length C8-C18) or laurylamidopropyl betaine (lauryl is an alkyl radical of chain length C12).

Hydroxysultaines which may be included in the composition may be selected from dodecylhydroxysultaine, tetradecylhydroxysultaine, hexadecylhydroxysultaine, laurylamidopropyl hydroxysultaine and cocamidopropyl hydroxysultaine.

Preferably the zwitterionic surfactant comprises at least one of laurylamidopropyl betaine, cocamidopropyl betaine, laurylamidopropyl hydroxysultaine and cocamidopropyl hydroxysultaine

The composition may comprise at least 3.5 wt % zwitterionic surfactant, preferably at least 4 wt %, more preferably at least 4.5 wt %, particularly preferably at least 5 wt % wherein the wt % is on the basis of the total weight of the composition. The composition may comprise at most 6.5 wt % zwitterionic surfactant, preferably at most 6 wt %, more preferably at most 5.5 wt %, wherein the wt % is on the basis of the total weight of the composition.

### Ratios of Components

The weight ratio of the sophorolipid surfactant to the anionic surfactant in the composition of the invention is from 1:20 to 1:2. This is advantageous because the synergy between the zwitterionic surfactant and the sophorolipid surfactant means a smaller relative amount of sophorolipid is required to improve the mildness of the anionic surfactant.

The weight ratio of the sophorolipid surfactant to the anionic surfactant in the composition may be at least 1:18, preferably at least 1:15, more preferably at least 1:12. The weight ratio of the sophorolipid surfactant to the anionic surfactant in the composition may be at most 1:2.5, preferably at most 1:3, more preferably at most 1:3.1, particularly at most 1:4. Preferably the weight ratio of the sophorolipid surfactant to the anionic surfactant in the composition is from 1:20 to 1:3.1.

Preferably the weight ratio of the sophorolipid surfactant to the zwitterionic surfactant in the composition is from 1:8 to 1:1.5. The weight ratio of the sophorolipid surfactant to the zwitterionic surfactant in the composition may be at least 1:7, preferably at least 1:6, more preferably at least 1:5. The weight ratio of the sophorolipid surfactant to the zwitterionic surfactant in the composition may be at most 1:2, preferably at most 1:2.5, more preferably at most 1:3.

The weight ratio of the zwitterionic surfactant to the anionic surfactant in the composition may be at least 1:10, preferably at least 1:6, more preferably at least 1:4. The weight ratio of the zwitterionic surfactant to the anionic surfactant in the composition may be at most 1:1, preferably at most 1:1.5.

### Use According to the Invention

The invention provides the use of a combination of a sophorolipid surfactant and an zwitterionic surfactant selected from the group consisting of betaines and hydroxysultaines in a composition to improve the mildness to skin of the composition by reducing the amount of zein protein solubilised by the composition, measured as described herein, wherein the composition comprises an anionic surfactant.

Preferably the composition comprises no free fatty acid. Preferably the composition comprises less than 5 wt % fatty acid, preferably comprises less than 4 wt % fatty acid, preferably comprises less than 3 wt % fatty acid, preferably comprises less than 2 wt % fatty acid, preferably comprises less than 1 wt % fatty acid, all on the basis of the total weight of the composition. Alternatively, the composition may comprise less than 25 grams of free fatty acid per litre (g/L), preferably less than 20 g/L, preferably less than 15 g/L, preferably less than 10 g/L on the basis of the total weight of free fatty acid and the total volume of the composition.

Preferably the anionic surfactant does not comprise a glycinate, a sulphosuccinate or mixtures thereof. Preferably the composition is a personal care formulation or home care formulation, more preferably a personal care formulation.

Preferably the composition is a hair care formulation which comprises at least one additional hair care ingredient.

Preferably the composition is a skin care formulation which comprises at least one additional skin care ingredient.

Preferably the amount of zein protein solubilised by the composition is reduced by at least 20 wt %, more preferably reduced by at least 30 wt %, yet more preferably reduced by at least 40 wt %, measured as described herein, when compared with an equivalent composition without the sophorolipid surfactant and the zwitterionic surfactant.

The use of the invention may comprise any of the other aspects or features of the invention described herein.

### Method According to the Invention

The invention provides a method of reducing the amount of zein protein solubilised by a composition comprising an anionic surfactant, the method comprising the steps of:
a) including a sophorolipid surfactant in the composition; and
b) including a zwitterionic surfactant selected from the group consisting of betaines and hydroxysultaines in the composition;
wherein the amount of zein protein solubilised by the composition is reduced by at least 20%, measured as described herein.

Preferably the amount of zein protein solubilised by the composition is reduced by at least 30 wt %, more preferably reduced by at least 40 wt %, measured as described herein, when compared with an equivalent composition without the sophorolipid surfactant and the zwitterionic surfactant.

The method of the invention may comprise any of the other aspects or features of the invention described herein.

### Personal Care and Home Care Formulations

A personal care formulation or home care formulation may comprise a composition according to the invention. The formulation may comprise no free fatty acid.

The home care formulation may be selected from fabric cleaners, fabric conditioners, stain removers, laundry detergents, hard surface cleaners, dishwashing detergents, machine dishwashing detergents, polishes and floor finishes.

The invention may provide a personal care formulation comprising the composition of the invention and a personal care ingredient. Preferably the personal care formulation is for topical application to skin or hair. The personal care formulation may be a hair care formulation comprising a composition according to the invention and at least one additional hair care ingredient. The personal care formulation may be a skin care formulation comprising a composition according to the invention and at least one additional skin care ingredient.

The personal care ingredient may be selected from a cleaning agent, hair conditioning agent, hair styling agent, anti-dandruff agent, hair growth promoter, perfume, sunscreen, sunblock, pigment, moisturizer, film former, hair color, make-up agent, thickening agent, emulsifier, humectant, emollient, antiseptic agent, deodorant active, dermatologically acceptable carrier, surfactant, abrasive, absorbent, fragrance, colorant, essential oil, astringent, anti-acne agent, anti-caking agent, anti-foaming agent, anti-oxidant, binder, enzyme, enzyme inhibitor, enzyme activator, coenzyme, botanical extract, ceramide, buffering agent, bulking agent, chelating agent, cosmetic biocide, external analgesic, substantivity increasing agent, opacifying agent, pH adjuster, reducing agent, sequestrant, skin bleaching and/or lightening agent, skin conditioning agent, skin soothing and/or healing agent, skin treating agent, vitamin or preservative. Preferably the personal care ingredient is selected from a cleaning agent, hair conditioning agent, skin conditioning agent, hair styling agent, antidandruff agent, hair growth promoter, perfume, sunscreen compound, pigment, moisturizer, film former, humectant, alpha-hydroxy acid, hair colour, make-up agent, thickening agent, antiseptic agent, deodorant, surfactant.

The hair care ingredient may be selected from shine enhancers, moisturisers, herbal additives, hair strengtheners, vitamin additives, colorants, hair thickening agents, setting and styling agents, hair cleaning agents, hair conditioning agents, anti-dandruff agents, hair growth promoters, hair color agents.

Preferably the skin care ingredient is selected from a skin cleaning agent, skin bleaching and/or lightening agent, skin conditioning agent, skin soothing and/or healing agent, skin treating agent.

The personal care formulations of the type defined herein may be in the form of emulsions (such as oil in water emulsions or water in oil emulsions); anhydrous formulations (such as hair oils, hair sprays/serums); detergent formulations; preferably oil in water emulsions and/or detergent formulations. Personal care emulsion formulations can take the form of pastes, creams, liquids and milks desirably, and in the field of hair care formulations aim to provide a pleasant aesthetic feel to the hair as well as improving manageability and visual appearance.

The personal care (or hair or skin care) formulation may have a range of different consistencies and/or viscosities depending on the desired end use of the formulation.

The end use applications of such formulations include, in the field of personal care products, moisturizers, body butters, gel creams, high perfume containing products, perfume creams, hair conditioners, hair relaxer formulations, hair shampoos, hair styling products, leave-on hair products, water-free products, anti-perspirant and deodorant products, cleansers, 2-in-1 foaming emulsions, emulsifier free products, mild formulations, scrub formulations e.g. containing solid beads, silicone in water formulations, pigment containing products, sprayable emulsions such as hair detanglers, colour cosmetics, shower products, make-up remover, eye make-up remover, and wipes. More preferably, the end use applications of such formulations include hair conditioners, hair relaxer formulations, hair shampoos, hair styling products, leave-on hair products and sprayable emulsions such as hair detanglers.

Preferably, the personal care (or hair or skin care) formulation is acidic. Preferably the formulation has a pH of between 1 and 6, preferably between 2 and 5.5, more preferably of between 3 and 5, and most preferably of between 4 and 4.8.

The personal care (or hair or skin care) formulation may comprise additional components, for example, one or more emulsifiers, emollients, carriers, surfactants and the like.

Preferably, the personal care (or hair or skin care) formulation further comprises an emulsifier. Preferably, the emulsifier is a non-ionic, high HLB (hydrophilic/lipophilic balance) surfactant which is capable of forming an oil-in-water emulsion. The emulsifier may be naturally derived. Examples of suitable emulsifiers include ethoxylated sorbitan esters, ethoxylated glyceryl esters, ethoxylated fatty alcohols (including lanolin alcohols), ethoxylated fatty acids (including lanolin fatty acids), glycerol fatty acid mono-esters, glycol fatty acid mono and di-esters, sugar esters (fatty acid mono and di esters of sucrose), fatty acid polyol (polyethylene glycol) esters, fatty alcohols (which may also act as co-emulsifiers), fatty acids and/or phosphate esters thereof, cationic surfactants or monoalkyl tertiary amines such as stearamidopropyl dimethylamine or behenamidopropyl dimethylamine.

The personal care (or hair or skin care) formulation may further comprise at least one co-emulsifier. Preferably, the or each co-emulsifier is a viscosity modifier, able to modify the viscosity of the formulation, more preferably a viscosity builder, able to increase the viscosity of the formulation. Preferably, the or each co-emulsifier is a fatty alcohol, preferably a C₁₂ to C₂₀ alcohol, more preferably a C₁₆ to C₁₈ alcohol, or a mixture thereof. Suitable alcohols for use as co-emulsifiers in the personal care formulation include cetyl alcohol, stearyl alcohol and cetearyl alcohol.

The formulations according to the present invention may also contain other additional emollient materials, preferably emollient oils. Preferably, the emollient oil is a non-polar oil. Examples of emollient oils which are suitable for use in the present formulation include mineral or paraffin oil; esters of fatty acids and fatty alcohols, preferably C₁₀-C₂₀ acids or alcohols, although isopropyl esters may be used; fatty acid glycol esters; fatty acid triglycerides; esters and diesters of alkoxylated fatty alcohols; botanical (plant) extracts; and hydrocarbons, preferably C₁₂-C₁₆. Preferably, the emollient is mineral oil. When present in the formulation, the or each additional emollient is preferably present at a concentration of at least 1% and up to 30% by weight based on the total weight of the formulation.

The personal care (or hair care) formulation according to the present invention may also contain one or more cationic ingredients. When present in the formulation, the or each cationic ingredient is preferably present at a concentration of between 0.01% and 10%, preferably between 0.05% and 8% and more preferably between 0.1% and 5% by weight based on the total weight of the formulation.

The personal care (or hair care) formulation according to the present invention may also contain one or more silicones. When present in the formulation, the or each silicone is preferably present at a concentration of between 0.05% and 10%, preferably between 0.1% and 8% and more preferably between 0.5% and 5% by weight based on the total weight of the formulation.

The personal care (or hair or skin care) formulation according to the present invention may also contain one or more film forming components. When present in the formulation, the or each film forming component is preferably present at a concentration of between 0.01% and 5%, preferably between 0.05% and 3% and more preferably between 0.1% and 2% by weight based on the total weight of the formulation.

Many other components may be used in the personal care (or hair or skin care) formulations according to the present invention. These components may be oil soluble, water soluble or non-soluble. Examples of such materials include:
(i) preservatives, preferably those approved for cosmetic use, particularly as listed in Annex 5 of the European Union cosmetics regulations. The preservative preferably comprises sodium benzoate, potassium sorbate or phenoxyethanol. The preservative may comprise parabens (alkyl esters of 4-hydroxybenzoic acid), substituted ureas or hydantoin derivatives, e.g. those sold commercially under the trade names Germaben II, Nipaguard BPX and Nipaguard DMDMH. The preservative may be used at a concentration in the range from 0.5 wt. % to 2 wt. % based on the total weight of the composition;
(ii) perfumes, when used typically at a concentration in the range from 0.1 wt. % to 10 wt. % more usually up to about 5 wt. % and particularly up to about 2 wt. %, based on the total weight of the composition;
(iii) humectants or solvents such as alcohols, polyols such as glycerol and polyethylene glycols, when used typically at a concentration in the range from 1 wt. % to 10 wt. % based on the total weight of the composition;
(iv) alpha hydroxy acids such as glycolic, citric, lactic, malic, tartaric acids and their esters; self-tanning agents such as dihydroxyacetone;
(v) vitamins and their precursors including: (a) Vitamin A, e.g. as retinyl palmitate and other tretinoin precursor molecules, (b) Vitamin B, e.g. as panthenol and its derivatives, (c) Vitamin C, e.g. as ascorbic acid and its derivatives, (d) Vitamin E, e.g. as tocopheryl acetate, (e) Vitamin F, e.g. as polyunsaturated fatty acid esters such as gamma-linolenic acid esters;
(vi) skin care agents such as ceramides either as natural materials or functional mimics of natural ceramides;
(vii) natural phospholipids, e.g. lecithin;
(viii) vesicle-containing formulations;
(ix) botanical extracts with beneficial skin care properties;
(x) skin whiteners such as kojic acid, arbutin and similar materials;
(xi) skin repair compounds actives such as Allantoin and similar series;
(xii) caffeine and similar compounds;
(xiii) cooling additives such as menthol or camphor;
(xiv) insect repellents such as N,N-diethyl-3-methylbenzamide (DEET) and citrus or eucalyptus oils;
(xv) essential oils; and
(xvi) pigments, including microfine pigments, particularly oxides and silicates, e.g. iron oxide, particularly coated iron oxides, and/or titanium dioxide, and ceramic materials such as boron nitride, or other solid components, such as are used in make-up and cosmetics, to give suspoemulsions, typically used in an amount in the range from 1 wt. % to 15 wt. %, but usually at least 5 wt. %, and particularly about 10 wt. % based on the total weight of the formulation.

The formulations may comprise a fragrance-imparting material to provide a pleasant scent. In one aspect, a scent is provided from a natural source, such as but not limited to alfalfa, almond, amber, angelica root, anise, apple, apricot, banana, basil, bay, bay laurel, benzoin, bergamot, bitter orange, black pepper, bois de rose (rosewood), cajeput, cardamom, carrot seed, cedarwood, cinnamon, citronella, citrus, clary sage, clove, cocoa, coconut, coffee, coriander, cranberry, cypress, elemi, eucalyptus globulous, eucalyptus, fennel, frankincense, galbanum, geranium, German chamomile, ginger, grapefruit, helichrysum, hyssop, jasmine, juniper berry, lavender, lemon, lemongrass, lily, linden blossom, mango, marjoram, melissa, mint, myrrh, myrtle, neroli, niaouli, nutmeg, orange, oregano, palm, parsley, patchouli, peach, peppermint, petitgrain, pine, pineapple, raspberry, Roman chamomile, rose, rosemary, sandalwood, spearmint, spruce, strawberry, tea, thyme, vanilla, vetiver, violet, yarrow, ylang, and the like. Preferably, the fragrance is selected from mint or vanilla.

The invention may provide a hair care formulation comprising the composition of the invention and a hair care ingredient. Preferably the hair care formulation is for topical application to hair.

Preferably the hair care formulation is a hair cleansing, conditioning, de-tangling, colour-protecting or styling formulation, particularly preferably a hair cleansing or conditioning formulation.

The hair care formulation may comprise a silicone fluid or oil such as dimethylpolysiloxane, dimethyl silicone, highly polymerised methyl polysiloxane, and methyl polysiloxane, known generically as dimethicone, cyclic oligomeric dialkylsiloxanes, such as the cyclic oligomers of dimethylsiloxane, known generically as cyclomethicone. The concentration of silicone oil in the formulation may preferably be in the range from 0.1 wt. % to 40 wt. %, more preferably 0.3 wt. % to 20 wt. %, particularly 0.5 wt. % to 5 wt. %, and especially 1 wt. % to 1.5 wt. % based on the total weight of the formulation. Alternatively, the hair care formulation may not comprise a silicone compound.

The hair care formulation may be in the form of an aqueous “leave on” or an aqueous “rinse off” end-use product. For such formulations, a dilute solution may be used.

Preferably, a buffered solution is used, in which the pH of the solution is adjusted to mildly acidic, with a pH in the range of from 4 to 6. In the case of rinse-off formulations, instructions are provided to wash off the diluted formulation after application. Depending on the level of treatment required, such instructions may also require the product to remain on the hair for some time, such as from 1 to 30 minutes. For leave-on formulations, the washing off step is omitted.

Where the formulation is a hair shampoo or conditioner which functions to make the hair straighter, the shampoo or conditioner may be in the form of a dispersion, emulsion or solution. One preferred system is one that forms liquid crystals. The liquid crystals are preferably lyotropic liquid crystals (i.e. both concentration and temperature dependent), more preferably lamellar phase liquid crystals, and particularly L alpha phase (neat) liquid crystals.

The hair care formulation may contain many different types of functional ingredients such as;
(i) cationic hair conditioning agents, e.g. ethoxylated phosphate fatty quats, such as those sold by Croda as Arlasilk™; fatty amido amines, such as those sold by Croda as Incromine™; fatty quats, such as those sold by Croda as Incroquat™, Crodazosoft™, Rejuvasoft™ or VibraRiche™ typically used at a concentration in the range from 1 wt. % to 5 wt. % based on the total weight of the composition. These are typically combined with polymeric hair conditioning cationic materials such as quaternised cellulose sold by Croda as Crodacel™ and MiruStyle™, quaternised proteins, such as those sold by Croda, as Croquat™, Crosilkquat™, Kerestore™ and Hydrotriticum™.
(ii) fatty alcohols, e.g. stearyl, cetearyl, cetyl, oleyl alcohols, used typically at a concentration range of 2 wt. % to 5 wt. % based on the total weight of the composition.
(iii) humectants or solvents, e.g. alcohols and polyols such as glycerol and polyethylene glycols, when used typically at a concentration in the range from 1 wt. % to 10 wt. % based on the total weight of the composition;
(iv) reconstructors, e.g. hydrolysed proteins such as wheat protein, which function to penetrate the hair and strengthen the hair structure through polymer crosslinking;
(v) glossing or detangling materials which bind to the hair and reflect light, e.g. silicones such as dimethicone, phenyltrimethicone, dimethiconol and/or trimethylsilylamodimethicone, usually at a concentration in the range from 0.2 wt. % to 10 wt. % based on the total weight of the composition;
(vi) acidity regulators, e.g. citric acid, lactic acid, which generally maintain the pH of the conditioner at about 4 to 6;
(vii) thermal protectors, usually heat-absorbing polymers, which shield the hair against excessive heat, e.g. caused by blow-drying or curling irons or hot rollers such as for instance those sold by Croda as Mirustyle™ MFP (quaternised starch); and
(viii) UV protection agents, to protect hair or formulation components from degradation by UV light, such as those sold by Croda as Crodasorb™ UV-HPP.

Any of the features described herein may be taken in any combination and with any aspect of the invention.

### EXAMPLES

The invention is illustrated by the following non-limiting examples. All parts and percentages are given by weight unless otherwise stated.

It will be understood that all tests and physical properties listed have been determined at atmospheric pressure and ambient/room temperature (i.e. about 23-25° C.), unless otherwise stated herein, or unless otherwise stated in the referenced test methods and procedures.

### Test Methods

In this specification the following test methods have been used:
(i) pH was measured at room temperature (25° C.) with a Fisher Scientific Accumet AE150 pH Meter. Depending on the initial pH reading, the pH of the formulation was adjusted to the range of 5.5-6.5 using 10 wt % sodium hydroxide solution (if the pH is higher) or 10 wt % citric acid solution (if the pH is lower). The pH was measured immediately and 24 hours after making the formulation.
(ii) Zein Solubilisation was measured as follows. Each surfactant was tested 5 times and an average was calculated:
1) Zein protein (70 mg) was dosed using the Mettler Toledo Quantos Powder Dispenser, into pre-weighed and labelled Eppendorf tubes.
2) Each surfactant solution (1 mL, 15%) was pipetted into an Eppendorf containing zein protein.
3) The Eppendorf tubes were agitated using a vortex mixer for 5 seconds and then placed on a roller to mix (1-hour, 60 rpm).
4) After mixing, each Eppendorf underwent centrifugation (5 mins, 20° C., 14800 rpm).
5) From each Eppendorf, supernatant was syringed out using a 1 mL syringe.
6) The Eppendorf tubes were placed in an oven at 50° C. for 24 hours with the lids open.
7) Eppendorf was weighed and amount of zein protein solubilised was calculated.

### Example 1

The composition of the sophorolipid sample (SL1) used in these examples is shown in Table 1 below. SL1 is partially hydrolysed after fermentation to give a 70:30 ratio by weight of acid form to lactone form sophorolipid. SL1 is produced using standard sophorolipid fermentation and production methods as are known in the art. A fermentation process using Starmerella bombicola with glucose and oleic acid as substrates is used. The fermentation and production is conducted so that no residual substrate is left in the SL1 product and the SL1 product consists essentially of sophorolipid and water.

TABLE 1
Sophorolipid composition of sample SL1

| | Concentration |
|---|---|
| Sample SL1 | (% w/w) |
| Mixed acylated & non-acylated acid | 70 |
| form sophorolipids (C16 to C18) | |
| Mixed acylated & non-acylated lactone | 30 |
| form sophorolipids (C16 to C18) | |

### Example 2

The following samples were made into 15 wt % active solutions by dilution in deionised water and the pH was adjusted to 6±0.8. Each sample was formulated immediately prior to testing hence, no preservative was added. Table 2 shows the samples made and active levels of all materials as supplied is shown to allow for appropriate dilutions to be made

TABLE 2
Making of 15 wt % active surfactant solutions

| | | Active Level |
|---|---|---|
| | | pre-dilution |
| Sample | INCI | (wt %) |
| SLES | Sodium Lauryl Ether Sulphate | 26 |
| SLSar* | Sodium Lauryl Sarcosinate | 30 |
| CAPS* | Cocamidopropyl Hydroxysultaine | 50 |
| CAPB* | Cocamidopropyl Betaine | 30 |
| Tween 28* | PEG-80 Sorbitan Laurate | 72 |
| SL1* | Sophorolipid | 40 |

*ex Croda

The term “active level” means the wt % of surface active component present in the commercial material containing the surfactant. Commercially available surfactants generally contain, in addition to the active material having surface-active properties, other components, such as for example water or solvents. The active level counts only the weight percentage of the surface active component.

### Example 3

Zein protein solubilisation provides an indication of the ability of a surfactant to solubilise the proteins within the top layer of the Stratum Corneum of human skin. This method was used to gain an understanding of the mildness of the surfactants in water to skin.

The percentage of zein protein solubilised by the solutions of 15 wt % active surfactant in water prepared in Example 2 was calculated based on the initial known mass of zein protein present compared to the final mass of the zein protein present after the solubilisation process. The results are shown in Table 3.

TABLE 3
Zein protein solubilised by individual surfactant solutions

| | | Zein Protein |
|---|---|---|
| Sample | INCI | Solubilised (wt %) |
| SLES | Sodium Lauryl Ether | 86 |
| | Sulphate | |
| SLSar* | Sodium Lauryl | 84 |
| | Sarcosinate | |
| CAPB* | Cocamidopropyl | 58 |
| | Betaine | |
| CAPS* | Cocamidopropyl | 30 |
| | hydroxysultaine | |
| SL1* | Sophorolipid | 1 |

*ex Croda

A high percentage of zein protein solubilised correlates to a harsh surfactant on the skin. Each solubilisation test was repeated 5 times per surfactant and an average calculated, along with the standard deviations between the five repeat measurements. It can be seen from the results that SL1 only solubilises 1% of zein protein and thus can be considered a very mild surfactant.

### Example 4

Sample SL1 has been identified as a very mild surfactant in Table 3. To investigate whether this material could perform as a mildness booster, reducing the irritancy of known harsher surfactants, increasing amounts of SL1 was added to 10% active surfactant solutions. Each surfactant solution was then tested for mildness using the zein solubilisation method. The results for the zein solubilisation of SL1 with SLES, SLS and CAPB at a 10% active level are shown in Tables 4, 5 & 6.

TABLE 4
Combination of SLES & SL1

| | Zein Protein |
|---|---|
| Sample | Solubilised (wt %) |
| 10% SLES & 0% SL1 | 86 |
| 10% SLES & 1% SL1 | 87 |
| 10% SLES & 3% SL1 | 77 |
| 10% SLES & 5% SL1 | 55 |
| 10% SLES & 10% SL1 | 5 |

TABLE 5
Combination of SLSar & SL1

| | Zein Protein |
|---|---|
| Sample | Solubilised (wt %) |
| 10% SLSar & 0% SL1 | 84 |
| 10% SLSar & 1% SL1 | 82 |
| 10% SLSar & 3% SL1 | 78 |
| 10% SLSar & 5% SL1 | 61 |
| 10% SLSar & 10% SL1 | 7 |

TABLE 6
Combination of CAPB & SL1

| | Zein Protein |
|---|---|
| Sample | Solubilised (wt%) |
| 10% CAPB & 0% SL1 | 58 |
| 10% CAPB & 1% SL1 | 32 |
| 10% CAPB & 3% SL1 | 1 |
| 10% CAPB & 5% SL1 | 1 |
| 10% CAPB & 10% SL1 | 1 |

Tables 4 to 6 show that sample SL1 significantly reduces the amount of zein protein solubilised when in a blend with SLES or SLSar at a concentration of 5% active and above. Almost no zein protein is solubilised when SL1 is present at a 10% active level or 1:1 ratio with the anionic surfactant. A similar result was also observed when SL1 was present in a surfactant solution with CAPB. However, only a 1% active addition of SL1 was required to significantly reduce the amount of zein protein solubilised and only 3% active addition was required to result in essentially no zein protein solubilisation. It must also be noted that during these experiments SL1 was added to the solution and did not replace the primary surfactant, hence with increasing concentration of SL1 the overall surfactant concentration of each solution also increased.

### Example 5

Example 4 showed that SL1 is effective at improving the mildness of SLES, SLSar and CAPB by reducing the amount of zein protein solubilised. For comparison, the effect of CAPB and Tween 28 on reducing the amount of zein protein solubilised by SLES is shown in Tables 7 & 8

TABLE 7
Combination of SLES & CAPB

| | Zein Protein |
|---|---|
| Sample | Solubilised (wt %) |
| 10% SLES & 0% CAPB | 91 |
| 10% SLES & 1% CAPB | 88 |
| 10% SLES & 3% CAPB | 76 |
| 10% SLES & 5% CAPB | 66 |

TABLE 8
Combination of SLES & Tween 28

| | Zein Protein |
|---|---|
| Sample | Solubilised (wt %) |
| 10% SLES & 0% Tween 28 | 91 |
| 10% SLES & 1% Tween 28 | 87 |
| 10% SLES & 3% Tween 28 | 80 |
| 10% SLES & 5% Tween 28 | 67 |
| 10% SLES & 10% Tween 28 | 21 |

Table 7 shows the impact on zein solubilisation with increasing concentration of CAPB to a 10% active SLES solution. Due to the increase in viscosity experienced when CAPB is added to SLES at high concentrations, this testing was limited to a 2:1 ratio of SLES:CAPB compared to a final 1:1 ratio which was used for SL1 (Table 4). The results show that CAPB reduces the amount of zein protein solubilised by SLES significantly when present at 3% active and above. However, at a 5% addition of CAPB, the zein protein solubilised is 66% compared to 55% when SL1 is added at the same concentration. Hence, SL1 is shown to reduce the irritancy of SLES more efficiently than CAPB, with respect to protein solubilisation. Table 8 shows a similar result was found when Tween 28 was investigated, with mildness boosting properties being observed but being less efficient than SL1.

### Example 6

From the results above it can be seen that SL1 is capable of significantly improving the mildness of a harsh surfactant solution, beyond that of any other benchmarks tested. It was investigated whether SL1 has any synergistic effects with other milder surfactants. The following study follows the same concept as previous but where the harsh surfactant solution is composed of a 10% active primary surfactant with 5% active CAPB and increasing concentrations of SL1. Hence the initial surfactant solution has a 15% active surfactant concentration which is increased with each addition of SL1. The primary surfactants investigated were SLES and SLSar and the results are shown in Tables 9 & 10.

TABLE 9
Combination of SLES & CAPB & SL1

| | Zein Protein |
|---|---|
| Sample | Solubilised (wt %) |
| 10% SLES & 5% CAPB & 0% SL1 | 63 |
| 10% SLES & 5% CAPB & 1% SL1 | 53 |
| 10% SLES & 5% CAPB & 3% SL1 | 9 |
| 10% SLES & 5% CAPB & 5% SL1 | 1 |
| 10% SLES & 5% CAPB & 10% SL1 | 1 |

TABLE 10
Combination of SLSar & CAPB & SL1

| | Zein Protein |
|---|---|
| | Solubilised |
| Sample | (wt %) |
| 10% SLSar & 5% CAPB & 0% SL1 | 80 |
| 10% SLSar & 5% CAPB & 1% SL1 | 77 |
| 10% SLSar & 5% CAPB & 3% SL1 | 15 |
| 10% SLSar & 5% CAPB & 5% SL1 | 1 |
| 10% SLSar & 5% CAPB & 10% SL1 | 1 |

What can be seen in Tables 9 & 10 is that a 5% active addition of CAPB to a 10% active solution of SLES, lowers the zein solubilisation percentage from 90% to 63%. However, this is not observed with SLSar, where the addition of CAPB does not have any impact on the mildness when added at this concentration. Surprisingly, it can be seen that with increasing concentration of SL1 the amount of zein protein solubilised is greatly reduced. In contrast to the previous tests, an addition of 3% SL1 with 5% CAPB reduces the amount of zein solubilisation by SLES/SLSar to such an extent that it provides a composition that is expected to be very mild to the skin. This can be seen to be a synergistic effect between CAPB & SL1 by comparing Tables 4, 7 & 9. Hence, these results show that SL1 works in synergy with CAPB to significantly reduce the irritancy of harsh surfactant formulations.

The experiments shown in Tables 9 & 10 were repeated including CAPS instead of CAPB and the results are given in Tables 11 & 12. The synergistic effect between CAPS & SL1 is greater than between CAPB & SL1 as shown in Tables 11 & 12.

TABLE 11
Combination of SLES & CAPS & SL1

| | Zein Protein |
|---|---|
| | Solubilised |
| Sample | (wt %) |
| 10% SLES & 5% CAPS & 0% SL1 | 80 |
| 10% SLES & 5% CAPS & 1% SL1 | 1 |
| 10% SLES & 5% CAPS & 3% SL1 | 1 |
| 10% SLES & 5% CAPS & 5% SL1 | 1 |
| 10% SLES & 5% CAPS & 10% SL1 | 1 |

TABLE 12
Combination of SLSar & CAPS & SL1

| | Zein Protein |
|---|---|
| Sample | Solubilised (wt %) |
| 10% SLSar & 5% CAPS & 0% SL1 | 79 |
| 10% SLSar & 5% CAPS & 1% SL1 | 55 |
| 10% SLSar & 5% CAPS & 3% SL1 | 1 |
| 10% SLSar & 5% CAPS & 5% SL1 | 1 |
| 10% SLSar & 5% CAPS & 10% SL1 | 1 |

### Example 7

Personal care and home care formulations may include sophorolipid SL1 of Example 1 as follows.

Table 13 shows an example of a hair care formulation in which the composition of the invention is included.

TABLE 13
Shampoo

| Ingredient (INCI) | Function | wt % |
|---|---|---|
| Water Deionised (Aqua) | Solvent | To |
| | | 100 |
| Pricerine ™ 9091 (Glycerin) | Humectant | 2 |
| SLES (Sodium Laureth Sulfate) | Anionic surfactant | 10 |
| Crodateric CAB30 (Cocamidopropyl | Amphoteric surfactant | 5 |
| Betaine) | | |
| SL1 (Sophorolipid) | Non-ionic surfactant | 3 |
| Sodium Chloride | Thickener | 1 |
| Cropeptide W (Aqua (and) Hydrolysed | Film forming & | 1 |
| Wheat Protein (and) Hydrolyzed Wheat | moisturising active | |
| Starch) | | |
| Phytessence Speedwell (Water (and) | Cleansing infusion | 1 |
| Glycerin (and) Veronica Officinalis | extract | |
| Flower/Leaf/Stem Extract) | | |
| Natragem S140 (Polyglyceryl-4 | Solubiliser | 2 |
| Laurate/Sebacate (and) Polyglyceryl-6 | | |
| Caprylate/Caprate (and) Aqua) | | |
| Cromollient SCE (Di-PPG-2 Myreth-10 | Emollient | 1 |
| Adipate) | | |
| Euxyl PE9010 (Phenoxyethanol (and) | Preservative | 0.5 |
| Ethylhexylglycerin) | | |

Table 14 shows an example of a skin care formulation in which the composition of the invention is included.

TABLE 14
Facial Cleanser

| Ingredient (INCI) | Function | wt % |
|---|---|---|
| Water Deionised (Aqua) | Solvent | To |
| | | 100 |
| Crodasinic LS30 (Water (and) Sodium | Non-ionic | 10 |
| Lauroyl Sarcosinate) | surfactant | |
| Crodateric CAS50 (Cocamidopropyl | Amphoteric | 5 |
| Hydroxysultaine) | surfactant | |
| SL1 (Sophorolipid) | Non-ionic | 3 |
| | surfactant | |
| Phytofoam (Water (and) Acacia Concinna | Foaming agent | 3 |
| Fruit Extract (and) Balanites Aegyptiaca | | |
| (Desert Date) Fruit Extract (and) Gypsophila | | |
| Paniculata Root Extract) | | |
| Arlasilk EFA (Linoleamidopropyl PG- | Moisturising | 2 |
| Dimonium Chloride Phosphate) | agent | |
| Arlasilk PTM (Myristamidopropyl PG- | Moisturising | 2 |
| Dimonium Chloride Phosphate) | agent | |
| Promidium LTS (PEG-150 Distearate (and) | Rheology | 3.5 |
| PPG-2 Hydroxyethyl Cocamide) | modifier | |
| Euxyl PE9010 (Phenoxyethanol (and) | Preservative | 0.5 |
| Ethylhexylglycerin) | | |

Table 15 shows an example of a home care formulation in which the composition of the invention is included.

TABLE 15
Hand Dishwash Formulation

| Ingredient | Function | wt % |
|---|---|---|
| Water | Solvent | 61.09 |
| Sodium Laureth Sulphate, 70% | Detergency | 13.90 |
| Sodium Lauryl Sulphate | Detergency | 5.51 |
| NatraSense AG-810* | Detergency, hydrotrope | 6.00 |
| Incromine ™ Oxide C* | Detergency | 2.00 |
| Cocamidopropyl Betaine | Detergency | 4.00 |
| SL1 | Detergency, mildness | 3.00 |
| Ethanol | Solvent | 3.00 |
| Citric Acid | Builder | 0.50 |
| Sodium Chloride | Viscosity modifier | 1.00 |

*ex Croda

It is to be understood that the invention is not to be limited to the details of the above embodiments, which are described by way of example only. Many variations are possible.

1. A combination of a sophorolipid surfactant and an zwitterionic surfactant selected from the group consisting of betaines and hydroxysultaines in a composition capable of improving the mildness to skin of the composition by reducing the amount of zein protein solubilised by the composition, wherein the composition comprises an anionic surfactant.

2. The combination according to claim 1 wherein the anionic surfactant does not comprise a glycinate, a sulphosuccinate or mixtures thereof.

3. The combination according to claim 1 wherein the composition is a personal care formulation or home care formulation.

4. The combination according to claim 1 wherein the composition is a hair care formulation which comprises at least one additional hair care ingredient.

5. The combination according to claim 1 wherein the composition is a skin care formulation which comprises at least one additional skin care ingredient.

6. The combination according to claim 1 wherein the amount of zein protein solubilised by the composition is reduced by at least 20 wt %, measured as described herein, when compared with an equivalent composition without the sophorolipid surfactant and the zwitterionic surfactant.

7. A method of reducing the amount of zein protein solubilised by a composition comprising an anionic surfactant, the method comprising the steps of:
a) including a sophorolipid surfactant in the composition; and
b) including a zwitterionic surfactant selected from the group consisting of betaines and hydroxysultaines in the composition;
wherein the amount of zein protein solubilised by the composition is reduced by at least 20 wt %, measured as described herein.

8. The method according to claim 7 wherein the composition does not comprise a glycinate surfactant, a sulphosuccinate surfactant, a fatty acyl isethionate or mixtures thereof.

9. The method according to claim 7 wherein the weight ratio of the sophorolipid surfactant to the anionic surfactant in the composition is from 1:20 to 1:3.1.

10. The method according to claim 7 wherein the weight ratio of the sophorolipid surfactant to the zwitterionic surfactant in the composition is from 1:8 to 1:1.5.

11. The method according to claim 7 wherein the composition is a personal care formulation or home care formulation.

12. The method according to claim 7 wherein the composition is a hair care formulation which comprises at least one additional hair care ingredient.

13. The method according to claim 7 wherein the composition is a skin care formulation which comprises at least one additional skin care ingredient.

14. A composition comprising:
a) sophorolipid surfactant;
b) anionic surfactant; and
c) 3 to 7 wt % zwitterionic surfactant selected from the group consisting of betaines and hydroxysultaines, wherein the wt % is on the basis of the total weight of the composition;
wherein the weight ratio of the sophorolipid surfactant to the anionic surfactant in the composition is from 1:20 to 1:2; and
wherein the composition comprises no free fatty acid.

15. The composition according to claim 14 wherein the weight ratio of the sophorolipid surfactant to the anionic surfactant in the composition is from 1:20 to 1:3.1.

16. The composition according to claim 14 wherein the weight ratio of the sophorolipid surfactant to the zwitterionic surfactant in the composition is from 1:8 to 1:1.5.

17. The composition according to claim 14 wherein the composition does not comprise a glycinate surfactant, a sulphosuccinate surfactant, a fatty acyl isethionate or mixtures thereof.

18. The composition according to claim 14 wherein the anionic surfactant comprises at least one of sodium lauryl sulphate, sodium lauryl ether sulphate and sodium lauryl sarcosinate.

19. The composition according to claim 14 wherein the anionic surfactant does not comprise a sulphate containing compound.

20. The composition according to claim 14 wherein the zwitterionic surfactant selected from the group consisting of betaines and hydroxysultaines comprises at least one of laurylamidopropyl betaine, cocamidopropyl betaine, laurylamidopropyl hydroxysultaine and cocamidopropyl hydroxysultaine.

21. The composition according to claim 14 wherein the sophorolipid surfactant comprises at least 55 wt % acid form sophorolipid and at most 45 wt % lactone form sophorolipid on the basis of the total weight of sophorolipid in the composition.

22. A personal care formulation or home care formulation comprising a composition according to claim 14.

23. A hair care formulation comprising a composition according to claim 14 and at least one additional hair care ingredient.

24. A skin care formulation comprising a composition according to claim 14 and at least one additional skin care ingredient.
